(19) United States
(12) Patent Application Publication
(10) Pub. No.: US 2020/0399698 A1
(43) Pub. Date: Dec. 24, 2020

(54) METHODS OF DETERMINING RESPONSE TO TNF ALPHA BLOCKERS

(71) Applicants: GENEFRON LTD., Har Adar (IL); YISSUM RESEARCH DEVELOPMENT COMPANY OF THE HEBREW UNIVERSITY OF JERUSALEM LTD., Jerusalem (IL)

(72) Inventor: Yoav SMITH, Jerusalem (IL)

(21) Appl. No.: 16/971,007

(22) PCT Filed: Feb. 19, 2019
(86) PCT No.: PCT/IL2019/050200

§ 371 Date: Aug. 19, 2020

Related U.S. Application Data
(60) Provisional application No. 62/632,280, filed on Feb. 19, 2018.

(51) Int. Cl. C12Q 1/6876

(52) U.S. Cl. CPC C12Q 1/6876; G01N 2800/102; G01N 2800/60; C12Q 2600/158; C12Q 2600/106

(57) ABSTRACT

Methods and kits for predicting the response of a subject to an anti-TNFα therapy, in high accuracy, are provided.

### CROSS REFERENCE TO RELATED APPLICATIONS

This application claims the benefit of priority of U.S. Provisional Patent Application No. 62/632,280, filed Feb. 19, 2018, the contents of which are all incorporated herein by reference in their entirety.

### FIELD OF INVENTION

The present invention is directed to, inter alia, methods and kits for gene expression profiling.

### BACKGROUND OF THE INVENTION

Rheumatoid arthritis (RA) is a common chronic autoimmune inflammatory disease affecting one percent of the population, leading to significant morbidity and mortality. Over the past two decades there have been major advances in therapy with the introduction of biologic agents. The use of these new therapies has made the current target of remission or low disease activity, an attainable goal. However, not all RA patients respond to biologic agents. When using the European League Against Rheumatism (EULAR) criteria, the incidence of remission in RA (Disease Activity Score 28 (DAS 28)<2.6) is less than 50% in most studies. Sixty percent of RA patients achieve a moderate or good EULAR response however, 30-40% of patients treated with a biologic, do not respond to the prescribed agent.

RA is a heterogeneous disease, and different biologic agents have different mechanisms of action. The preferred approach would be to target the chief mechanism at play in an individual's disease. Current clinical practice relies on clinical predictors, including DAS 28, Health Assessment Questionnaire (HAQ), use of concurrent DMARDS, gender as well as presence of rheumatoid factor (RF) or anti-cyclic citrullinated peptide (anti-CCP). However, these predictors do not account for the variance in clinical response and lead to successive trial and error of different biologics with considerable burden to the patient as well as significant economic consequences. In recent years there has been an effort to search for biomarkers to allow personalized medicine, however at present there are currently no clinically useful biomarkers to predict response of the individual patient to a specific biologic agent.

### SUMMARY OF THE INVENTION

The invention provides, is some embodiments, methods and kits for predicting the response of a subject to an anti-TNFα therapy, in high accuracy.

According to some embodiments, there is provided a method and a kit for determining a therapeutic response criterion in subject suffering from rheumatoid arthritis, such as to determine the EULAR criteria of the subject following TNFα blocker therapy.

According to a first aspect, there is provided a method for determining suitability to receive anti-TNFα therapy, in a subject in need thereof, the method comprising the step of:
a. determining an expression level sum of three genes in a biological sample obtained from the subject, wherein the three genes are
   i. MX1
   ii. at least one gene selected from IFI6 and OAS3, and
   iii. at least one gene selected from the group consisting of: IFI6, OAS1, OAS3, DDX58, RSAD2, IFIT1, IFIT3, IFI44, IFI44L, IFITM3, and HERC5; and
b. generating a diagnosis regarding suitability to receive anti-TNFα therapy wherein:
   an expression level sum above a predetermined threshold, is indicative of the subject being suitable to receive anti-TNFα therapy, and
   an expression level sum below a predetermined threshold, is indicative of the subject being unsuitable to receive anti-TNFα therapy,

thereby determining suitability of a subject to receive anti-TNFα therapy

According to some embodiments, the biological sample is peripheral blood mononuclear cell (PBMC), or whole blood.

According to some embodiments, the subject suffers from rheumatoid arthritis.

According to some embodiments, the anti-TNFα therapy is TNFα blocker therapy.

According to some embodiments, the third gene is selected from IFI6, OAS1, OAS3, DDX58, RSAD2, and HERC5. The method of any one of claims 1 to 5, wherein the three genes are MX1, IFI6 and OAS3.

According to some embodiments, the predetermined threshold is between 0.2-0.5.

According to some embodiments, the method further comprises treating the suitable subjects with anti-TNFα therapy.

According to some embodiments, the method further comprises transmitting the diagnosis to the subject.

According to another aspect, there is provided a kit comprising reagents adapted to specifically determine the expression level of MX1, and at least one of IFI6, and OAS3.

According to some embodiments, the kit further comprises reagents adapted to specifically determine the expression level of MX1, IFI6 and OAS3. According to some embodiments, the kit further comprises at least one reagent adapted to specifically determine the expression level of a gene selected from IFI6, OAS1, OAS3, DDX58, RSAD2, IFIT1, IFIT3, IFI44, IFI44L, IFITM3, and HERC5. According to some embodiments, the kit further comprises at least one reagent adapted to specifically determine the expression level of a gene selected from IFI6, OAS1, OAS3, DDX58, RSAD2, and HERC5.

According to some embodiments, the reagents are selected from nucleic acid hybridization or amplification reagents, and a plurality of nucleic acid-specific probes or amplification primers.

According to some embodiments, the kit further comprises any one of: (i) detectable tags or labels, (ii) solutions for rendering a nucleic acid susceptible to hybridization, (iii) solutions for lysing cells, (iv) solutions for the purification of nucleic acids, (v) any combination of (i), (ii), (iii), (iv) and (v).

According to some embodiments, the kit further comprises at least one reagent adapted to specifically determine the expression level of at least one house-keeping gene.

According to some embodiments, the kit consists of:
a. at least one reagent adapted to specifically determine the expression level of MX1
b. at least one reagent adapted to specifically determine the expression level of at least one gene selected from IFI6 and OAS3;
c. at least one reagent adapted to specifically determine the expression level of at least one gene selected from IFI6, OAS1, OAS3, DDX58, RSAD2, IFIT1, IFIT3, IFI44, IFI44L, IFITM3, and HERC5, wherein the gene is not the gene selected in (b); and
d. at least one reagent adapted to specifically determine the expression level of at least one house-keeping gene.

According to some embodiments, the house-keeping gene is a gene that is not differentially expressed between subjects that respond and subjects that do not respond to anti-TNFα therapy.

According to some embodiments, the kit consists of:
a. at least one reagent adapted to specifically determine the expression level of MX1
b. at least one reagent adapted to specifically determine the expression level of at least one gene selected from IFI6 and OAS3; and
c. at least one reagent adapted to specifically determine the expression level of at least one gene selected from IFI6, OAS1, OAS3, DDX58, RSAD2, IFIT1, IFIT3, IFI44, IFI44L, IFITM3, and HERC5, wherein the gene is not the gene selected in (b).

According to some embodiments, the kit consists of at least one reagent adapted to specifically determine the expression level of MX1, at least one reagent adapted to specifically determine the expression level of IFI6 and at least one reagent adapted to specifically determine the expression level of OAS3.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a volcano plot of Interferon Signature Gene (ISG) expression in RA patients: TNF blocker responders (n=6) vs. TNF blocker non-responders (n=13). Each dot in the plot represents the ratio of expression of a single gene in responders vs. non-responders. This figure shows a distinct and statistically significant difference in this ratio for 15 ISG genes: IFIT1, IFIT2, IFIT3, IFI44L, IFI44, MX1, HERC5, DDX60L, DDX60, OAS3, OAS1, IFIT5, IFIH1, IFI6, IFITM3 enabling clear differentiation between TNFα blocker responders and non-responders.

FIG. 2 demonstrates multiple interactions between TNF and the IFN related genes using STRING database. Several of the 12 selected genes are marked in this figure (ISG15, IF16, MX1, IFIT1, IFT3) for demonstration. Red nodes depict first shell of interactors, white nodes depict second shell of interactors.

FIG. 3 is a bar graph depicting prediction response of 19 patients with RA treated with Infliximab.

FIG. 4 is a bar graph depicting prediction of RA patients' response to a TNFα blocker utilizing the EULAR moderate response criteria using expression levels of MX1, IFI6, HERC5 and OAS1.

FIG. 5 is a bar graph depicting prediction of RA patients' response to a TNFα blocker utilizing the EULAR good response criteria using expression levels of MX1, IFI6, and OAS3.

FIG. 6 is a ROC curve and reverse ROC curve of prediction with the 12 genes.

FIG. 7A is a bar graph of gene expression in whole blood from 8 RA patients. NR=non-responders, R=responders.

FIG. 7B is a bar graph of gene expression in PBMCs from 8 RA patients. NR=non-responders, R=responders.

FIG. 7C is a bar graph of the difference in expression in whole blood of seven genes between TNFα blocker responders and non-responders.

FIG. 7D is a bar graph of the difference in expression in PBMCs of seven genes between TNFα blocker responders and non-responders.

FIG. 7E is a bar graph of the combined expression of MX1, IFI6 and OAS3 in whole blood from 8 RA patients. NR=non-responders, R=responders.

FIG. 7F is a bar graph of the difference in expression in PBMCs of seven genes between TNFα blocker responders and non-responders. NR=non-responders, R=responders.

FIG. 7G is a bar graph of the combined expression of MX1, IFI6 and OAS3 in whole blood from 10 RA patients. NR=non-responders, R=responders.

### DETAILED DESCRIPTION OF THE INVENTION

The invention provides, is some embodiments, methods and kits for predicting the response of a rheumatoid arthritis (RA) patient to a TNFα blocker, in high accuracy.

As exemplified herein. in order to detect the most appropriate genes that might play a role in response to TNFα blocker treatment, gene expression of patients with RA treated with TNFα blockers was examined utilizing published microarray data sets as well as examining new patient samples. The methods and kits disclosed herein are based, in part, on gene expression of selected genes detected in these data sets, and analyzed under two separate analyses, a retrospective and a prospective analysis.

Surprisingly, accurate prediction of a good EULAR response could be determined by analyzing expression levels of a specific subset of 12 genes: IFIT1, IFIT3, IFI44, IFI44L, IFITM3, IFI6, OAS1, OAS3, HERC5, MX1, RSAD2 and DDX58. Although each gene had a predictive value, the combination of all 12 was superior to any one individual gene. Unexpectedly, an even smaller subset of just three genes, MX1, IFI6, and OAS3, when combined was capable of standing in for the full set of 12 and accurately predicting patient response to TNFα blockers. In particular MX1 was found to be the most robust and accurate prediction gene, followed by OAS3 and then IF16.

According to one aspect, there is provided a method for determining a therapeutic response criterion to TNFα blocker, in a subject suffering from rheumatoid arthritis, the method comprising the step of:
determining an expression level of MX1, IFI6, and at least one antigen selected from the group consisting of: OAS1, OAS3, and HERC5, in a biological sample obtained from the subject;
wherein:
increased expression levels of MX1, IFI6, and at least one antigen selected from the group consisting of: OAS1, OAS3, and HERC5, compared to control, is indicative of the subject being a responder to TNFα blocker therapy, and
decreased expression levels of MX1, IFI6, and at least one antigen selected from the group consisting of: OAS1, OAS3, and HERC5, compared to control, is indicative of the subject being a non-responder to TNFα blocker therapy.

According to another aspect, there is provided a method for determining a therapeutic response criterion to TNFα blocker, in a subject suffering from rheumatoid arthritis, the method comprising the step of:
determining an expression level of MX1, IFI6, and at least one antigen selected from the group consisting of: OAS1, OAS3, and HERC5, in a biological sample obtained from the subject;
wherein:
increased expression levels of MX1, IFI6, and OAS3, compared to control, are indicative of the subject being a good responder to TNFα blocker therapy,
increased expression levels of MX1, IFI6, HERC5 and OAS1, compared to control, are indicative of the subject being a moderate responder to TNFα blocker therapy, and
decreased expression levels of MX1, IFI6, and at least one antigen selected from the group consisting of: OAS1, OAS3, and HERC5, compared to control, is indicative of the subject being a non-responder to the TNFα blocker therapy;
thereby determining the therapeutic response criterion of a subject suffering from rheumatoid arthritis.

According to another aspect, there is provided a method for determining suitability to receive anti-TNFα therapy, in a subject in need thereof, the method comprising the step of:
determining an expression level sum of three genes in a biological sample obtained from the subject, wherein said three genes are
   i. MX1
   ii. at least one gene selected from IFI6 and OAS3, and
   iii. at least one gene selected from the group consisting of: IFI6, OAS1, OAS3, DDX58, RSAD2, IFIT1, IFIT3, IFI44, IFI44L, IFITM3, and HERC5; and
wherein:
an expression level sum above a predetermined threshold, is indicative of the subject being suitable to receive anti-TNFα therapy, and
an expression level sum below a predetermined threshold, is indicative of the subject being unsuitable to receive anti-TNFα therapy,

thereby determining suitability of a subject to receive anti-TNFα therapy.

In some embodiments, said biological sample is peripheral blood mononuclear cell (PBMC). In some embodiments, said biological sample is whole blood. In some embodiments, said biological sample is selected from PBMCs and whole blood. In some embodiments, the sample is from a routine blood draw. In some embodiments, the RNA is collected by Tempus RNA isolation.

In some embodiments, the method comprises generating a diagnosis regarding suitability to receive anti-TNFα therapy. In some embodiments, the diagnosis is automatically generated. In some embodiments, gene expression is calculated using an automated device. In some embodiments, gene expression is calculated via polymerase chain reaction (PCR). In some embodiments, gene expression is calculated by microarray. In some embodiments, the automated device is a PCR machine. In some embodiments, the diagnosis is transmitted to the subject. In some embodiments, the method further comprises transmitting the diagnosis to the subject. In some embodiments, the transmitting is automatic. In some embodiments, the transmitting is electronic.

In some embodiments, said expression levels is a sum of expression levels. In some embodiments, expression of at least 2 genes are summed. In some embodiments, at least 3 genes are summed. In some embodiments, expression values are normalized by comparison to a house-keeping gene or an internal control. As used herein, a “house-keeping gene” is an internal control gene that is expressed in all cells being analyzed and whose expression is representative of the number of the size of the sample. In some embodiments, the size is the number of cells in the sample. In some embodiments, the size the amount of RNA in the sample. In some embodiments, house-keeping gene expression is unaltered between test groups. In some embodiments, house-keeping gene expression is unaltered between test groups of equal size. In some embodiments, house-keeping gene expression is unaltered between the anti-TNFα therapy responders and non-responders. Thus, a gene that has an expression that is altered in one group or the other (in the responder/non-responders for example) would be excluded from being a house-keeping gene. In some embodiments, a house-keeping gene is a gene that is not differentially expressed between subject that respond and subjects that do not respond to anti-TNFα therapy. Examples of house-keeping genes include, but are not limited to GAPDH, beta-actin, and ribosomal RNA. In some embodiments, the house-keeping gene is GAPDH.

In some embodiments, the subject suffers from an inflammatory disease. In some embodiments, the subject suffers from an autoimmune disease. In some embodiments, the subject suffers from arthritis. In some embodiments, the subject suffers from rheumatoid arthritis (RA). In some embodiments, the arthritis is selected from RA, psoriatic arthritis, and juvenile chronic arthritis. In some embodiments, the subject suffers from a disease or condition treatable with anti-TNFα therapy. In some embodiments, the subject suffers from a disease selected from RA, inflammatory bowel disease, Crohn's disease, psoriatic arthritis, juvenile chronic arthritis, psoriasis, and ankylosing spondylitis.

In some embodiments, said control is a pre-determined threshold. In some embodiments, said pre-determined threshold is between 0.2-0.5. In some embodiments, said pre-determined threshold is between 0.2-0.4. In some embodiments, said pre-determined threshold is between 0.3-0.5. In some embodiments, said pre-determined threshold is between 0.3-0.4. In some embodiments, the threshold quantifies relative expression as compared to a house-keeping gene. In some embodiments, the house-keeping gene is GAPDH. Thus, expression above a threshold of 0.2, for example, refers to expression that is more than 20% of the expression of the house-keeping gene, i.e. GAPDH. In some embodiments, the pre-determined threshold is determined using the expression quantifying techniques described herein. In some embodiments, the pre-determined threshold is per a specific quantity of input. In some embodiments, the pre-determined threshold is for PBMC samples. In some embodiments, the pre-determined threshold is for whole blood samples. In some embodiments, the pre-determined threshold is higher for whole blood vs PCMC samples. In some embodiments, the pre-determined threshold is for 6-7 ml of whole blood extracted from a subject. In some embodiments, the pre-determined threshold is for PBMCs extracted from 6-7 ml of whole blood extracted from a subject. In some embodiments, 6-7 ml is 6 ml.

The quantification is expressed as the change in expression levels of mRNA interpreted as complementary DNA (cDNA, generated by reverse transcription of mRNA). Relative quantification is easier to carry out as it does not require a calibration curve as the amount of the studied gene is compared to the amount of a control reference gene.

As the units used to express the results of relative quantification are unimportant the results can be compared across a number of different PCRs. The reason for using one or more housekeeping genes is to correct non-specific variation, such as the differences in the quantity and quality of RNA used, which can affect the efficiency of reverse transcription and therefore that of the whole PCR process. However, it is crucial that the reference gene be stable.

In some embodiments, the method further comprises treating the suitable subjects with anti-TNFα therapy. In some embodiments, the method further comprises selecting an alternative therapy for subjects unsuitable for anti-TNFα therapy. Alternative therapies are well known in the art and any such alternative therapy may be applied to unsuitable subjects as determined by the method of the invention. Examples of alternative non-TNFα therapy include, but are not limited to abatacept, rituximab, or tocilizumab.

In some embodiments, the third gene is selected from IFI6, OAS1, OAS3, DDX58, RSAD2, and HERC5. In some embodiments, the three genes are MX1, IFI6 and OAS3. In some embodiments, the second gene is IFI6. In some embodiments, the third gene is OAS3.

According to another aspect, there is provided a kit comprising reagents adapted to specifically determine the expression level of a plurality of antigens selected from the group consisting of: MX1, IFI6, OAS3, HERC5 and OAS1. According to some embodiments, there is provided a kit comprising reagents adapted to specifically determine the expression level of a set of antigens selected from the group consisting of: MX1, IFI6, OAS3, HERC5 and OAS1.

By another aspect, there is provided a kit comprising reagents adapted to specifically determine the expression level of MX1, and at least one gene selected from IFI6 and OAS3. In some embodiments, the kit comprising reagents adapted to specifically determine the expression level of MX1 and IFI6. In some embodiments, the kit comprising reagents adapted to specifically determine the expression level of MX1 and OAS3. In some embodiments, the kit further comprises at least one reagent adapted to specifically determine the expression level of a gene selected from IFI6, OAS1, OAS3, DDX58, RSAD2, IFIT1, IFIT3, IFI44, IFI44L, IFITM3, and HERC5. In some embodiments, the kit further comprises at least one reagent adapted to specifically determine the expression level of a gene selected from IFI6, OAS1, OAS3, DDX58, RSAD2, and HERC5. In some embodiments, the kit further comprises at least one reagent adapted to specifically determine the expression level of a gene selected from IFI6, OAS1, and OAS3. In some embodiments, the kit further comprises at least one reagent adapted to specifically determine the expression level of a gene selected from IFI6, HERC5, and OAS3. In some embodiments, the kit further comprises at least one reagent adapted to specifically determine the expression level of a gene selected from IFI6, DDX58, and OAS3. In some embodiments, the kit further comprises at least one reagent adapted to specifically determine the expression level of a gene selected from IFI6, RSAD2, and OAS3.

In some embodiments, the kit comprises reagents adapted to specifically determine the expression level of MX1, IFI6 and OAS3. In some embodiments, the kit further comprises at least one reagent adapted to specifically determine the expression level of a gene selected from OAS1, DDX58, RSAD2, IFIT1, IFIT3, IFI44, IFI44L, IFITM3, and HERC5. In some embodiments, the kit further comprises at least one reagent adapted to specifically determine the expression level of a gene selected from OAS1, DDX58, RSAD2, and HERC5.

In some embodiments, the kit further comprises at least one reagent adapted to specifically determine the expression level of at least one house-keeping gene. In some embodiments, the house keeping gene is GAPDH. In some embodiments, the house-keeping gene is not differentially expressed between subjects that respond and subjects that do not respond. In some embodiments, the house-keeping gene does not alter expression in response to interferon.

In some embodiments, said reagents are selected from nucleic acid hybridization or amplification reagents, and a plurality of nucleic acid-specific probes or amplification primers. In some embodiments, the kit further comprising any one of: (i) detectable tags or labels, (ii) solutions for rendering a nucleic acid susceptible to hybridization, (iii) solutions for lysing cells, (iv) solutions for the purification of nucleic acids, (v) any combination of (i), (ii), (iii), (iv) and (v).

In some embodiments, the kit consists of:
a. at least one reagent adapted to specifically determine the expression level of MX1
b. at least one reagent adapted to specifically determine the expression level of at least one gene selected from IFI6 and OAS3;
c. at least one reagent adapted to specifically determine the expression level of at least one gene selected from IFI6, OAS1, OAS3, DDX58, RSAD2, IFIT1, IFIT3, IFI44, IFI44L, IFITM3, and HERC5, wherein said gene is not the gene selected in (b); and
d. at least one reagent adapted to specifically determine the expression level of at least one house-keeping gene.

In some embodiments, the kit consists of:
a. at least one reagent adapted to specifically determine the expression level of MX1
b. at least one reagent adapted to specifically determine the expression level of at least one gene selected from IFI6 and OAS3; and
c. at least one reagent adapted to specifically determine the expression level of at least one gene selected from IFI6, OAS1, OAS3, DDX58, RSAD2, IFIT1, IFIT3, IFI44, IFI44L, IFITM3, and HERC5, wherein said gene is not the gene selected in (b).

In some embodiments, the kit consists of at least one reagent adapted to specifically determine the expression level of MX1, at least one reagent adapted to specifically determine the expression level of IFI6 and at least one reagent adapted to specifically determine the expression level of OAS3. In some embodiments, the kit consists of at least one reagent adapted to specifically determine the expression level of MX1, at least one reagent adapted to specifically determine the expression level of IFI6, at least one reagent adapted to specifically determine the expression level of OAS3 and at least one reagent adapted to specifically determine the expression level of at least one house-keeping gene.

In some embodiments, at least one reagent of the kit of the invention is attached to a solid support. In some embodiments, at least one reagent of the kit of the invention is attached to an artificial support. In some embodiments, the reagents of the kit of the invention are attached to a chip and/or array. In some embodiments, the kit provides an array for use in determining suitability of a subject to receive anti-TNFα therapy. In some embodiments, the kit is for use in determining suitability of a subject to receive anti-TNFα therapy. In some embodiments, the kit consists of an array with the above described reagents.

As known to one skilled in the art, the EULAR (European League Against Rheumatism) response criteria is a classified response criteria which classifies the patients individual as non-responders, moderate responders or good responders, dependent on the change and the level of the Disease Activity Score (DAS) and the Disease Activity Score 28 (DAS28).

In some embodiments, increased expression levels of MX1, IFI6, and OAS3, compared to a threshold value, is indicative of the subject being a good responder to TNFα blocker therapy. In some embodiments, the sum levels of MX1, IFI6, and OAS3, compared to a threshold value, is indicative of the subject being a good responder to TNFα blocker therapy.

In some embodiments, a sum expression levels of MX1, IFI6, and OAS3, above 0.2, above 0.3, above 0.4 or above 0.5 is indicative of the subject being a good responder to TNFα blocker therapy. Each possibility represents a separate embodiment of the present invention.

In some embodiments, a sum expression levels of MX1, IFI6, and OAS3, below 0.2, below 0.3, below 0.35, or below 0.4 is indicative of the subject being a non-responder to TNFα blocker therapy. Each possibility represents a separate embodiment of the present invention.

In some embodiments, increased expression levels of MX1, IFI6, HERC5 and OAS1, compared to a threshold value, is indicative of the subject being a moderate responder to TNFα blocker therapy. In some embodiments, the sum levels of MX1, IFI6, HERC5 and OAS1, compared to a threshold value, is indicative of the subject being a moderate responder to TNFα blocker therapy.

In some embodiments, a sum expression levels of MX1, IFI6, HERC5 and OAS1, above 0.2, above 0.25, above 0.3, above 0.35, above 0.4 or above 0.5 is indicative of the subject being a moderate responder to TNFα blocker therapy. Each possibility represents a separate embodiment of the present invention.

In some embodiments, a sum expression levels of MX1, IFI6, HERC5 and OAS1, below 0.2 is indicative of the subject being a moderate responder to TNFα blocker therapy. Each possibility represents a separate embodiment of the present invention.

In another embodiment, the invention provides a method for determining the efficacy of a TNFα blocker, for treating RA in a subject in need thereof, the method comprising determining the expression levels of MX1, IFI6, and OAS3 in a sample obtained from the subject, wherein a sum expression levels of above 0.4 or above 0.5, indicates a good EULAR response in the subject.

In another embodiment, the invention provides a method for determining the efficacy of a TNFα blocker, for treating RA in a subject in need thereof, the method comprising determining the expression levels of MX1, IFI6, HERC5 and OAS1 in a sample obtained from the subject, wherein a sum expression levels of above 0.2, above 0.25, above 0.3, above 0.35, above 0.4 or above 0.5, indicates a moderate EULAR response in the subject.

In some embodiments, the anti-TNFα therapy is TNFα blocker therapy. In one embodiment, said therapy is TNFα blocker therapy. The term “TNFα blocker” includes agents which interfere with TNFα activity. The term also includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356. In one embodiment, the TNFα inhibitor used in the invention is an anti-TNFα antibody, or a fragment thereof, including infliximab (Remicade®, Johnson and Johnson; described in U.S. Pat. No. 5,656,272), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380. In another embodiment, the TNFα inhibitor is a TNF fusion protein, e.g., etanercept (Enbrel®, Amgen; described in WO 91/03553 and WO 09/406,476, incorporated by reference herein). In another embodiment, the TNFα inhibitor is a recombinant TNF binding protein (r-TBP-I) (Serono).

In some embodiments, the TNFα blocker therapy is selected from the group consisting of Etanercept. Adalimumab, and Golimumab.

Myxovirus (influenza virus) resistance 1 (MX1) gene (GenBank Accession No. NM_002462, NM_001178046, NM_001144925) encodes the MX1 protein (GenBank Accession No. NP_002453 NP_001171517, NP_001138397).

Interferon, alpha-inducible protein 6 (IFI6) gene (GenBank Accession Nos. NM_022873; NM_022872; NM_002038) encodes the IFI6 protein (GenBank Accession Nos. NP_075011; NP_075010; NP_002029).

Interferon induced protein with tetratricopeptide repeats 1 (IFI1T) gene (GenBank Accession Nos. NM_001548; NM_001270927; NM_001270928, NM_001270929, NM_001270930) encodes the IFIT1 protein (GenBank Accession Nos. NP_001539; NP_001257856; NP_001257857, NP_001257858, NP_001257859).

Interferon induced protein with tetratricopeptide repeats 3 (IFIT3) gene (GenBank Accession Nos. NM_001549; NM_001031683; NM_001289758, NM_001289759) encodes the IFIT3 protein (GenBank Accession Nos. NP_001540; NP_001026853; NP_001276687, NP_001276688).

Interferon induced protein with tetratricopeptide repeats 4 (IFIT4) gene (GenBank Accession Nos. AF_083470) encodes the IFIT4 protein (GenBank Accession Nos. AAC_63524). In some embodiments, IFIT4 is known as interferon induced tetratricopeptide protein IFI60.

Interferon induced protein 44 like (IFI44L) gene (GenBank Accession Nos. NM_006820, XM_005270391, XM_005270392, XM_005270393, XM_005270394, XM_017700120, XM_024452685) encodes the IFI44L protein (GenBank Accession Nos. NP_006811, XP_005270448, XP_005270449, XP_005270450, XP_006710367, XP_016855609, XP_024308453). In some embodiments, IFIT4 is known as interferon induced tetratricopeptide protein IFI60.

Interferon induced transmembrane protein 3 (IFITM3) gene (GenBank Accession No. NM_021034, NR 049759) encodes the IFITM3 protein (GenBank Accession No. NP_066362).

HECT and RLD domain containing E3 ubiquitin protein ligase 5 (HERC5) gene (GenBank Accession No. NM_016323) encodes the HERC5 protein (GenBank Accession No. NP_057407).

2′-5′-oligoadenylate synthetase 3 (OAS3) gene (GenBank Accession No. NM_006187) encodes the OAS3 protein (GenBank Accession No. NP_006178.2).

2′-5′-oligoadenylate synthetase 1 (OAS1) gene (GenBank Accession No. NM_016816, NM_002534, NM_001032409) encodes the OAS1 protein (GenBank Accession No. NP_058132, NP_002525, NP_001027581).

DExD/H-box helicase 58 (DDX58) gene (GenBank Accession No. NM_014314) encodes the DDX58 protein (GenBank Accession No. NP_055129).

Radical S-adenosyl methionine domain containing 2 (RSAD2) gene (GenBank Accession No. NM_080657, XM_011510415) encodes the RSAD2 protein (GenBank Accession No. NP_542388, XP_011508717).

Glyceraldehyde-3-phosphate dehydrogenase (GAPDH) gene (GenBank Accession No. NM_002046, NM_001256799, NM_001289745, NM_001289746, NM_001289743, NR_152150) encodes the GAPDH protein (GenBank Accession No. NP_002037, NP_001243728, NP_001276674, NP_001276675, NP_001276672).

It should be noted that the terms “sensitivity” and “specificity” are used herein with respect to the ability of a subset of markers, to correctly classify a sample as belonging to a pre-established population associated with responsiveness to treatment with a certain medicament, e.g., to determine a therapeutic response criterion of a subject suffering from rheumatoid arthritis. In one embodiment, the kit and method provided herein has a sensitivity of at least 80%, at least 85%, at least 90%, at least 95%, at least 99%, or 100% sensitivity. In one embodiment, the kit and method provided herein has a specificity of at least 65%, at least 70%, at least 75%, at least 80% specificity.

The term “accuracy”, as used herein, means a statistical measure for the correctness of classification or identification of sample types. The accuracy is the proportion of true results (both true positives and true negatives). In one embodiment, the kit and method provided herein has an accuracy of at least 80%, at least 85%, at least 90%, or at least 95%, accuracy.

In some embodiments, the contacting is performed in-vitro or ex-vivo. In some embodiments, the method of the invention is performed in-vitro or ex-vivo,

In some embodiments, the method further comprises the step of comparing the expression profile to a reference expression profile (such of a healthy control, or a population of RA subjects undergoing a specific therapy). In some embodiments, the method further comprises normalization of the expression values. In some embodiments, the normalization is performed with an internal control.

In some embodiments, the plurality of antigens comprises at least 3, 4, 5 antigens. Each possibility represents a separate embodiment of the present invention. In some embodiments, the plurality of antigens is at most 6, 7, 8, 9, 10, 11, 12, 13, 14, 15, 16, 17, 18, 19, 20, 21, 22, 23, 24, 25, 26, 27, 28, 29, 30, antigens. Each possibility represents a separate embodiment of the present invention. In some embodiments, the required plurality for high diagnosis accuracy varies for different applications (the different sets of expression profiles) of the methods of the invention. In some embodiments, an antigen is a gene.

As used herein, the term “subject” refers to any mammal, including both human and other mammals. In some embodiments, the methods of the present invention are applied to human subjects.

In some embodiments, the biological sample is obtained from a subject suspected to be affected by RA. Suitable samples include, but are not limited to, a cell, cell lysate, a protein sample, tissue, homogenized tissue, organ, homogenized organ, and bodily fluid. As will be appreciated by a skilled artisan, the method of collecting and preparing a sample from a subject can and will vary depending upon the nature of the sample. Any of a variety of methods generally known in the art may be utilized to collect a sample. Generally speaking, the method preferably maintains the integrity of the nucleic acid molecules such that they can accurately be detected and/or quantified in the sample.

In some embodiments, the biological sample is a bodily fluid including but not limited to whole blood, serum, plasma, cerebrospinal fluid, saliva, urine, spinal fluid, abdomen fluid, breast milk and lymphocyte or cell culture supernatants. In some embodiments, the biological sample is selected from: blood, whole blood, plasma, serum and fractions thereof. In some embodiments, the biological sample is selected from: PBMC (peripheral blood mononuclear cell), erythrocytes, leukocytes or thrombocytes. In some embodiments, the biological sample is PBMC.

The term “expression” generally refers to the process by which gene-encoded information is converted into the structures present and operating in the cell. For example, biomarker gene expression values measured in Real-Time Polymerase Chain Reaction, sometimes also referred to as RT-PCR or quantitative PCR (qPCR), represent luminosity measured in a tested sample, where an intercalating fluorescent dye is integrated into double-stranded DNA products of the qPCR reaction performed on reverse-transcribed sample RNA, i.e., test sample RNA converted into DNA for the purpose of the assay. The luminosity is captured by a detector that converts the signal intensity into a numerical representation which is said expression value, in terms of miRNA. Therefore, according to the invention “expression” of a gene, specifically, a gene encoding the biomarker genes of the invention may refer to transcription into a polynucleotide. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. Methods for determining the level of expression of the biomarkers of the invention will be described in more detail herein after.

The terms “level of expression” or “expression level” are used interchangeably and generally refer to a numerical representation of the amount (quantity) of a polynucleotide which encodes an amino acid product or protein in a biological sample.

The term “expression profile” refers to expression of a group/set of genes. In some embodiments, the expression profile may be detected at the expression levels such as by analyzing and determining RNA values (e.g., mRNA or miRNA). The RNA levels may be determined in various samples.

As used in reference with the methods of the invention, “increase in expression of the expression profile” refers to a sum increase of expression of the specific biomarker set provided herein. For a non-limiting example, a specific value of increase may be a result of increase of all the antigens of the set. Alternatively, specific value of increase may be a result of increase of only a few antigens of the set. In some embodiments, the increase refers to at least 50% increase, 60% increase, 70% increase, 80% increase, 90% increase, 100% increase in expression level of the expression profile.

In some embodiments, the step of determining the level of expression by the method of the invention further comprises an additional and optional step of normalization. According to this embodiment, in addition to determination of the level of expression of the biomarkers of the invention, the level of expression of at least one suitable control reference gene (e.g., housekeeping genes) is being determined in the same sample. According to such embodiment, the expression level of the biomarkers of the invention obtained in step (a) is normalized according to the expression level of said at least one reference control gene obtained in the additional optional step in said test sample, thereby obtaining a normalized expression value. Optionally, similar normalization is performed also in at least one control sample or a representing standard when applicable.

The term “expression value” thus refers to the result of a calculation, that uses as an input the “level of expression” or “expression level” obtained experimentally and by normalizing the “level of expression” or “expression level” by at least one normalization step as detailed herein, the calculated value termed herein “expression value” is obtained. As used herein, “normalized values” may be the quotient of raw expression values of marker genes, divided by the expression value of a control reference gene from the same sample, such as a stably-expressed housekeeping control gene. Any assayed sample may contain more or less biological material than is intended, due to human error and equipment failures Importantly, the same error or deviation applies to both the marker genes of the invention and to the control reference gene, whose expression is essentially constant. Thus, division of the marker gene raw expression value by the control reference gene raw expression value yields a quotient which is essentially free from any technical failures or inaccuracies (except for major errors which destroy the sample for testing purposes) and constitutes a normalized expression value of said marker gene. This normalized expression value may then be compared with normalized cutoff values, i.e., cutoff values calculated from normalized expression values. In certain embodiments, the control reference gene may be a gene that maintains stable in all samples analyzed in the microarray analysis.

It should be noted that normalized biomarker genes expression level values that are higher (positive) or lower (negative) in comparison with a corresponding predetermined standard expression value or a cut-off value in a control sample predict to which population of patients the tested sample belongs.

It should be appreciated that in some embodiments an important step in determining the expression level is to examine whether the normalized expression value of any one of the biomarker genes of the tested sample is within the range of the expression value of a standard population or a cutoff value for such population.

More specifically, the specific expression values of the tested samples are compared to a predetermined cutoff value. As used herein the term “comparing” denotes any examination of the expression level and/or expression values obtained in the samples of the invention as detailed throughout in order to discover similarities or differences between at least two different samples. It should be noted that comparing according to the present invention encompasses the possibility to use a computer-based approach.

In some embodiments, the method of the invention refers to a predetermined cutoff value. It should be noted that a “cutoff value”, sometimes referred to simply as “cutoff” herein, is a value that meets the requirements for both high diagnostic sensitivity (true positive rate) and high diagnostic specificity (true negative rate).

In certain alternative embodiments, a control sample may be used (instead of, or in addition to, pre-determined cutoff values). Accordingly, the normalized expression values of the biomarker genes used by the invention in the test sample are compared to the expression values in the control sample. In certain embodiments, such control sample may be obtained from at least one of a healthy subject, a subject suffering from the same pathologic disorder, a subject that responds to treatment with said medicament and a non-responder subject.

The term “response” or “responsiveness” to a certain treatment refers to an improvement in at least one relevant clinical parameter as compared to an untreated subject diagnosed with the same pathology (e.g., the same type, stage, degree and/or classification of the pathology), or as compared to the clinical parameters of the same subject prior to interferon treatment with said medicament.

The term “non-responder” to treatment with a specific medicament, refers to a patient not experiencing an improvement in at least one of the clinical parameter and is diagnosed with the same condition as an untreated subject diagnosed with the same pathology (e.g., the same type, stage, degree and/or classification of the pathology), or experiencing the clinical parameters of the same subject prior to treatment with the specific medicament.

The rate of change in the expression value of the different marker genes of the invention may reflect either reduction or elevation of expression. More specifically, “reduction” or “down-regulation” of the marker genes as a result of interferon treatment includes any “decrease”, “inhibition”, “moderation”, “elimination” or “attenuation” in the expression of said genes and relate to the retardation, restraining or reduction of the biomarker genes expression or levels by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

Alternatively, “up-regulation” of any one of the biomarker genes as a result of interferon or any other drug treatment includes any “increase”, “elevation”, “enhancement” or “elevation” in the expression of said genes and relate to the enhancement and increase of at least one of the biomarker genes expression or levels by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

As appreciated, a predetermined rate of change calculated for a pre-established population as detailed above for example encompasses a range for the rate of change having a low value and a high value, as obtained from a population of individuals including healthy controls, responders and non-responders to said medicament. Thus, a subgroup of responsive patients can be obtained from the entire tested population. In this pre-established responsive population, the low value may be characterized by a low response whereas the high value may be associated with a high response as indicated by regular clinical evaluation. Therefore, in addition to assessing responsiveness to treatment, the rate of change may provide insight into the degree of responsiveness. For example, a calculated rate of change that is closer in its value to the low value may be indicative of a low response and thus although the patient is considered responsive, increasing dosing or frequency of administration may be considered. Alternatively, a calculated rate of change that is closer in its value to the high value may be indicative of a high response, even at times leading to remission and thus lowering the administration dosage may be considered.

For clarity, when referring to a pre-established population associated with responsiveness, or the ability to eradicate pathogens, it is meant that a statistically-meaningful group of patients treated with a specific medicament was analyzed as disclosed herein, and the correlations between the biomarker gene/s expression values (and optionally other patient clinical parameters) and responsiveness to such treatment was calculated. The population may optionally be further divided into sub-populations according to other patient parameters, for example gender and age.

A variety of known techniques may be suitable for determining an expression profile. Such techniques include methods based on hybridization analysis of polynucleotides and on sequencing of polynucleotides, and proteomics-based methods. In some embodiments, the determining step is performed by nucleic acid hybridization, nucleic acid amplification, or an immunological method. In some embodiments, the determining step is performed in-situ. In some embodiments, fluorescence labeling or staining are applied. In some embodiment, an imaging step is further applied.

In some embodiments, the expression, and the level of expression, of proteins or polypeptides of interest can be detected through immunohistochemical staining of tissue slices or sections. Additionally, proteins/polypeptides of interest may be detected by Western blotting, ELISA or Radioimmunoassay (RIA) assays employing protein-specific antibodies.

Alternatively, protein levels can be determined by constructing an antibody microarray in which binding sites comprise immobilized, preferably monoclonal, antibodies specific to a plurality of proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art.

In some embodiments, the determining step comprises the step of obtaining nucleic acid molecules from said non-testis biological sample. In some embodiments, the nucleic acids molecules are selected from mRNA molecules, DNA molecules and cDNA molecules. In some embodiments, the cDNA molecules are obtained by reverse transcribing the mRNA molecules. In some embodiments, the expression profile is determined by measuring mRNA levels of the antigens. Methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995).

Numerous methods are known in the art for measuring expression levels of a one or more gene such as by amplification of nucleic acids (e.g., PCR, isothermal methods, rolling circle methods, etc.) or by quantitative in situ hybridization. Design of primers for amplification of specific genes is well known in the art, and such primers can be found or designed on various web sites such as http://bioinfo.ut.ee/primer3-0.4.0/ or https://pga.mgh.harvard.edu/primerbank/ for example.

The skilled artisan will understand that these methods may be used alone or combined. Non-limiting exemplary method are described herein.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in the tissue as compared to comparable tissues.

RNA-Seq: RNA-Seq uses recently developed deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts, or assembled de novo without the genomic sequence to produce a genome-scale transcription map that consists of both the transcriptional structure and/or level of expression for each gene. To avoid artifacts and biases generated by reverse transcription direct RNA sequencing can also be applied.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed by commercially available equipment, following manufacturer's protocols, such as by using the Affymetrix GenChip technology, or Incyte's microarray technology.

As used herein, the terms “amplification” or “amplify” mean one or more methods known in the art for copying a target nucleic acid, e.g., the genes listed in any of the Tables disclosed herein, thereby increasing the number of copies of a selected nucleic acid sequence. Amplification may be exponential or linear. In a particular embodiment, the target nucleic acid is RNA.

As used herein, “nucleic acid” refers broadly to segments of a chromosome, segments or portions of DNA, cDNA, and/or RNA. Nucleic acid may be derived or obtained from an originally isolated nucleic acid sample from any source (e.g., isolated from, purified from, amplified from, cloned from, or reverse transcribed from sample DNA or RNA).

As used herein, the term “oligonucleotide” refers to a short polymer composed of deoxyribonucleotides, ribonucleotides or any combination thereof. Oligonucleotides are generally between about 10 and about 100 nucleotides in length. Oligonucleotides are typically 15 to 70 nucleotides long, with 20 to 26 nucleotides being the most common. An oligonucleotide may be used as a primer or as a probe. An oligonucleotide is “specific” for a nucleic acid if the oligonucleotide has at least 50% sequence identity with a portion of the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, or at least 95% sequence identity.

As used herein, a “fragment” in the context of a nucleic acid refers to a sequence of nucleotide residues which hare at least about 5 nucleotides, at least about 7 nucleotides, at least about 9 nucleotides, at least about 11, nucleotides, or at least about 17, nucleotides. A fragment is typically less than about 300 nucleotides, less than about 100 nucleotides, less than about 75 nucleotides less than about 50 nucleotides, or less than about 30 nucleotides. In certain embodiments, the fragments can be used in polymerase chain reaction (PCR), or various hybridization procedures to identify or amplify identical or related DNA molecules.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

As used herein, “target nucleic acid” refers to segments of a chromosome, a complete gene with or without intergenic sequence, segments or portions a gene with or without intergenic sequence, or sequence of nucleic acids to which probes or primers are designed. Target nucleic acids may be derived from genomic DNA, cDNA, or RNA. As used herein, target nucleic acid may be native DNA or a PCR-amplified product.

The expression data used in the methods disclosed herein may be normalized. The term “normalized” with regard to a gene transcript or a gene expression product refers to the level of the transcript or gene expression product relative to the mean levels of transcripts/products of a set of reference genes, wherein the reference genes are either selected based on their minimal variation across, patients, tissues or treatments (“housekeeping genes”), or the reference genes are the totality of tested genes.

In general, samples may be normalized by a common factor. For example, cell-containing samples are normalized by protein content or cell count. In some embodiments, samples (i.e., the expression levels) are normalized using a set of normalization genes. In another embodiment, said expression levels are normalized expression levels. With respect to RT-PCR experiments involving archived fixed paraffin embedded tissue samples, sources of systematic variation are known to include the degree of RNA degradation relative to the age of the patient sample and the type of fixative used to store the sample. Other sources of systematic variation are attributable to laboratory processing conditions.

Assays may provide normalization by incorporating the expression of certain normalizing genes, which do not differ significantly in expression levels under the relevant conditions. Exemplary normalization genes include housekeeping genes. Alternatively, or additionally, array datasets can be normalized using known RMA, MAS 5.0, Z scoring and by reference to their average values.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); “Bacteriophage Methods and Protocols”, Volume 1: Isolation, Characterization, and Interactions, all of which are incorporated by reference. Other general references are provided throughout this document. Various embodiments and aspects of the present invention as delineated hereinabove and as claimed in the claims section below find experimental support in the following examples. Reference is now made to the following examples, which together with the above descriptions illustrate some embodiments of the invention in a non-limiting fashion.

### Materials and Methods

### Gene Detection for Kit Development

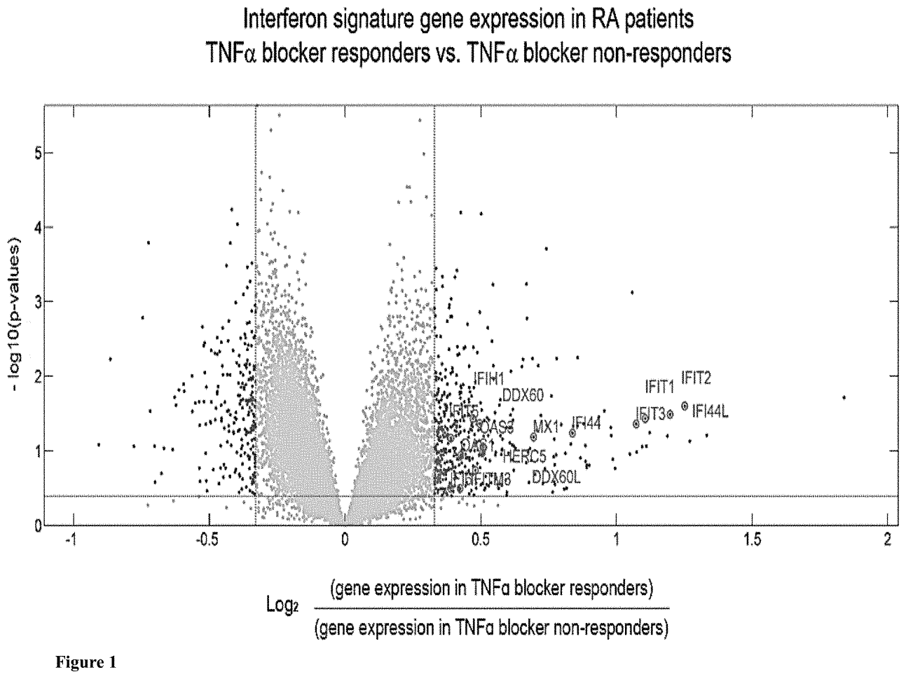

Gene expression of patients with RA treated with TNFα blockers were examined utilizing published microarray data sets. Volcano plots, which indicate the p value and differential ratio of each gene (responders vs. non-responders) in relation to all other expressed genes were used. The analysis clearly demonstrated the central role of 15 genes belonging to the interferon pathway in the prediction of response to TNFα blocker treatment (FIG. 1). Of the 15 detected genes, the 12 most consistent and biologically relevant genes were chosen for use in the kit disclosed herein. The selected genes include IFN signature genes (ISG), and a downstream ubiquitin group of genes. The kit was constructed based on the selected group of genes and a unique algorithm.

### Role of Selected Genes in RA

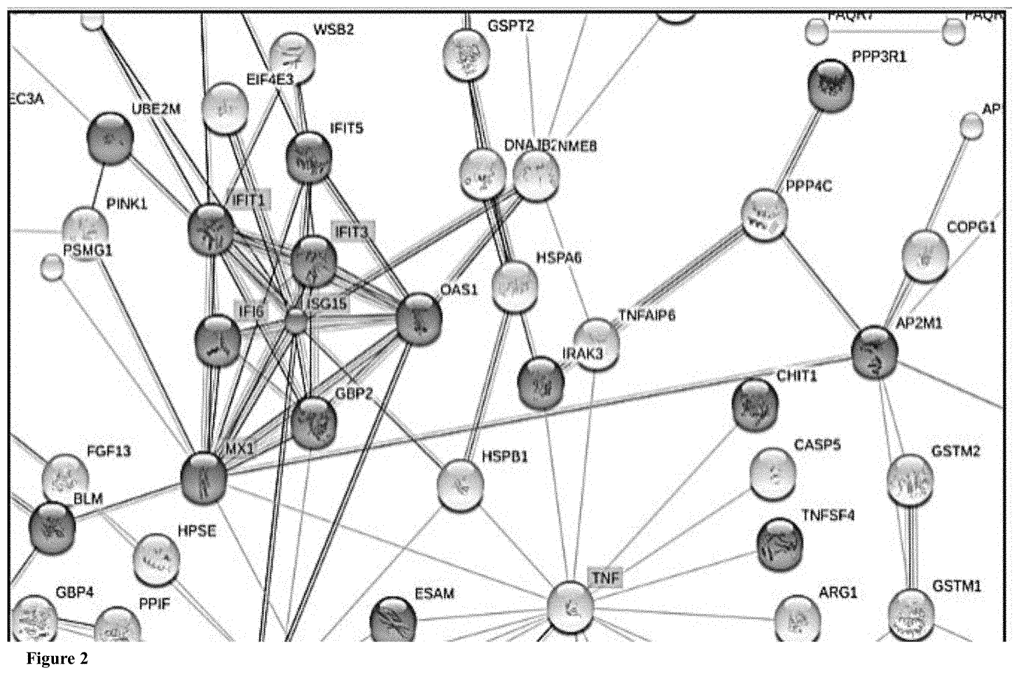

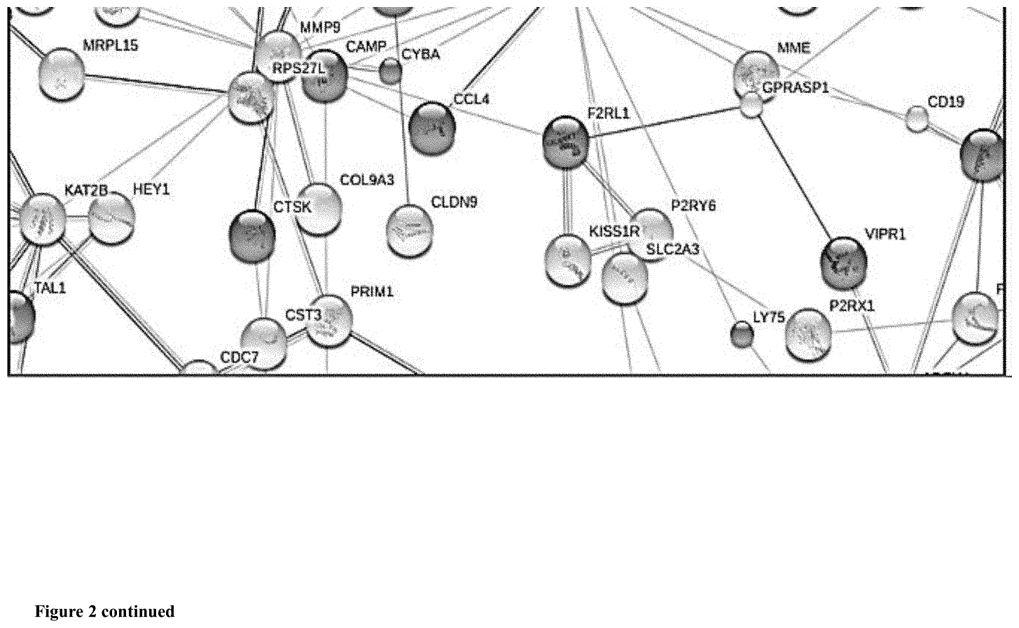

STRING is a database of known and predicted protein-protein interactions. We uploaded into the STRING database a list of 400 genes shown to be significantly upregulated (more than 2-fold) in RA (n=105) as compared to controls (n=20) (p<0.0001). When uploading these genes into the STRING database, a strong association of the selected genes with TNF was demonstrated, supporting their selection for the kit and method of the invention (FIG. 2).

The selected genes used for the kit include IFN genes and a downstream ubiquitin group of genes. The biologic validity for use of these genes is further supported when looking at any normalized dataset of RNA gene expression of IFN genes and the ubiquitin group of genes. These genes consistently demonstrate the highest correlated values to one another, verifying their established pathway resulting in activation via the Interferon sensitive response element (ISRE) common promoter.

### Patients

For the retrospective analysis, we utilized two published patient data sets published by Toonen et al. (Validation study of existing gene expression signatures for anti-TNF treatment in patients with rheumatoid arthritis. PLoS One. 2012; 7: e33199) and Mesko et al. (Peripheral blood derived gene panels predict response to infliximab in rheumatoid arthritis and Crohn's disease. Genome Med. 2013; 5:59). For the prospective analysis, consecutive patients with active RA were recruited from the department of Rheumatology at the Tel-Aviv medical Center. The procedures followed were in accordance with the ethical standards of the responsible committee on human experimentation and with the Declaration of Helsinki. The study was approved by the local ethics committee board. All patients signed an informed consent. Inclusion criteria for this study included: age >18 years, a definite diagnosis of RA based on the 2010 ACR/EULAR criteria for RA (Aletaha D, et al. 2010. Arthritis & Rheum 2010; 62:2569-2581), active RA defined by DAS 28 ESR>3.2, designation for treatment with a TNFα blocker as prescribed by the treating physician. According to Israeli health insurance prescribing guidelines all patients designated to receive a biologic agent had received previous treatment with at least three disease-modifying anti-rheumatic drugs (DMARDs), including methotrexate.

### Clinical Assessment

All patients in the prospective analysis were evaluated before starting treatment with a TNFα blocker. Patients were re-evaluated three months after starting treatment. Assessment included swollen joint count (SJC)-66 joints, tender joint count (TJC)-68 joints, patient assessment of pain (visual analogue scale of 100 mm), patient global assessment (visual analogue scale of 100 mm), physician global assessment (visual analogue scale of 100 mm), health assessment questionnaire (HAQ), ESR and CRP. Response to therapy was defined according to the EULAR response criteria where a patient had achieved a moderate EULAR response if the DAS score had decreased by >0.6 but less than 1.2. A good EULAR response had been achieved if the DAS score had decreased by at least 1.2.

### Gene Expression Assessment

Blood samples: A blood sample (6-7 ml for PBMC extraction, or 3 ml for whole blood) was withdrawn from each patient before administration of a TNFα blocker.

### Pbmc Preparation:

PBMCs were prepared within 2 hours from blood collection and were stabilized with RNA Later. Each whole blood sample was layered carefully over Ficoll-Hypaque gradient (Novomed Uni-Sep U-04) and centrifuged for 25 minutes at room temperature. The middle buffy coat layer was collected and diluted with 10 ml of PBS and centrifuged at 4° C. The cell pellet was re-suspended in 1 ml of PBS and centrifuged at 4° C. The supernatant was discarded, and the cells were re-suspended in 250 μl of RNAlater solution (AM 7020 Life technologies). Each sample was stored at 2-8° C. for 24 hours and then stored at −700 C.

### qRT PCR:

Total RNA was purified from PBMCs by using RNAqueous® Kit (AM 1912 Life Technology) according to kit instructions and stored at −70° C. until use. The RNA preparations were converted into cDNA by using High Capacity cDNA Reverse Transcription Kit (AB-4374966 Life Technology) according to the company's instructions. For gene expression measurements, the Quant Studio 12K Flex PCR system (Life Technology) was used where the reaction mix contained: Taq Man Gene Expression Master Mix (2×) 5 and 12 ng CDNA in 0.4 μl H₂O and H₂O 4.1 μl. In parallel, the ‘no template control’ (NTC) contained the same components without cDNA. A volume of 0.5 μl of Taq Man Gene Expression Assay (20×) enzyme was added to all samples, which were pre-mixed with the appropriate primers. Each primer was tested in triplicates. The PCR reactions were carried out under the following conditions: 2 min at 500 C, 10 min at 95° C. for denaturation and 40 cycles of 15 sec in 95° C. followed by 60° C. for 1 min. The following TaqMan probes were used for gene expression analysis: OAS3 (Hs00196324), DDX58 (Hs00204833), RSAD2 (Hs00369813), MX1 (Hs00895608), IFI6 (Hs00242571), HERC5 (Hs00180943), OAS1 (Hs00973637), GAPDH (Hs99999905). These probes are commercially available, such as from Thermo Fisher. Gene expression results are displayed as expression relative to GAPDH. Thus, an expression level of 0.5 indicates that the gene was expressed at a level that was half of GAPDH.

### Statistical Analysis:

Gene expression of 12 genes was measured using GAPDH as a normalizing gene. Three readings were obtained for each gene. Results obtained from the analysis of the first cases allowed differentiation of two groups: responders versus non-responders. The score for each patient consisted of the weighted sum of expression of the 12 selected genes: (IFIT1, IFIT3, IFI44, IFI44L, IFITM3, IFI6, OAS1, OAS3, HERC5, MX1, RSAD2 and DDX58) or the sum of 3 genes: MX1, IFI6 and OAS3. An extensive ROC analysis was then carried out in order to evaluate the statistical power of the selected genes to correctly differentiate TNFα blocker responders from non-responders. Prediction accuracy, prediction sensitivity and specificity were calculated.

### Example 1

### Retrospective Analysis

Using our pre-selected genes, we analyzed Toonen et al.'s data set of patients with RA and could correctly identify the response to a TNFα blocker in 23 of 24 non-responders and in 14 of 18 responders, resulting in an accurate prediction in 37 of 42 patients with RA treated with TNFα blockers (prediction accuracy—88%).

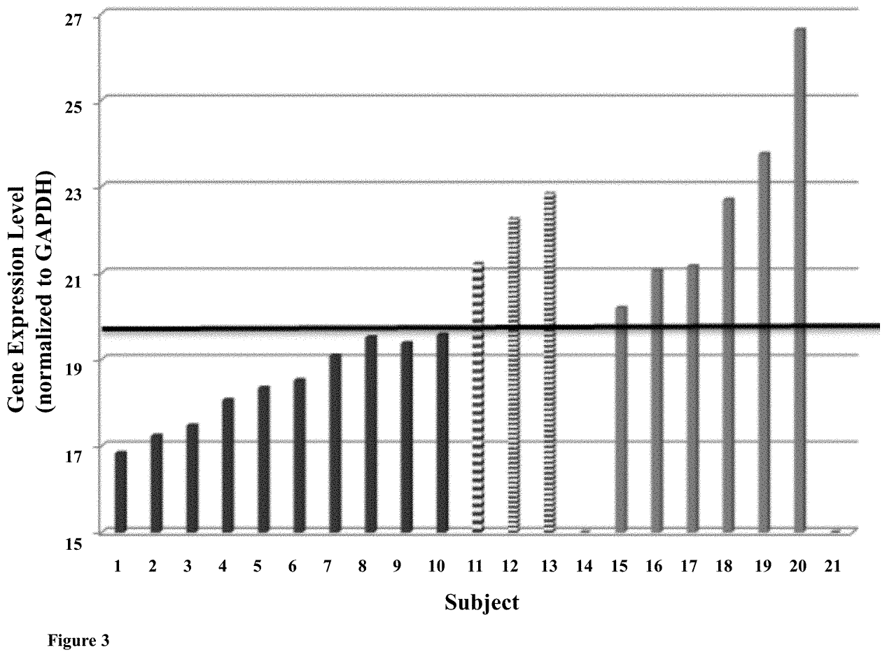

Applying the same pre-selected gene set, we analyzed another data set of 19 patients with RA treated with Infliximab, published by Mesko et al. and were able to correctly identify the response of all 6 responders and 10 of 13 non-responders with an accuracy of 84.2% (FIG. 3).

### Example 2

### Prospective Analysis

Eighteen patients with active RA were recruited for this study and were assessed before starting treatment with a TNFα blocker. Patient demographics and clinical parameters, including history of RA therapies as well as the type of TNFα blocker administered are presented in table 1.

TABLE 1
Patient demographics and clinical parameters

| | |
|---|---|
| Gender F/M | 18/0 |
| Age (mean) | 57.6 ± 12.3 |
| Disease duration | 10.5 ± 13.4 (1-58) |
| (mean, range) | |
| Seropositivity | 14/17 |
| (RF and/or anti-CCP) | |
| Previous | DMARDs (including MTX) - 17 |
| treatment | Tocilizumab-3 |
| | Etanercept-2 |
| | Adalimumab- 1 |
| | No prior biologic- 12 |
| Currently on corticosteroids | n = 7 |
| Prednisone dose | 5-12 mg/d (mean 3.87 ± 4.85) |
| TNF | Etanercept-11 |
| blocker | Adalimumab-5 |
| administered | Golimumab-1 |
| DAS at baseline | 5.26 ± 1.04 |
| DAS at 3 months | 3.91 ± 1.44 |
| Significance in DAS reduction | P = 0.0008 |
| at 3 months | |

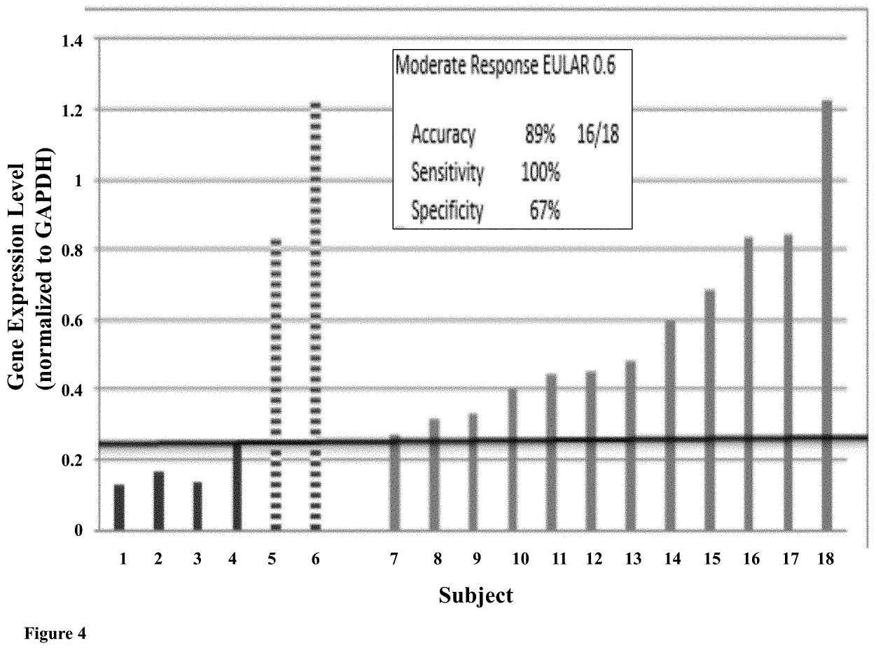

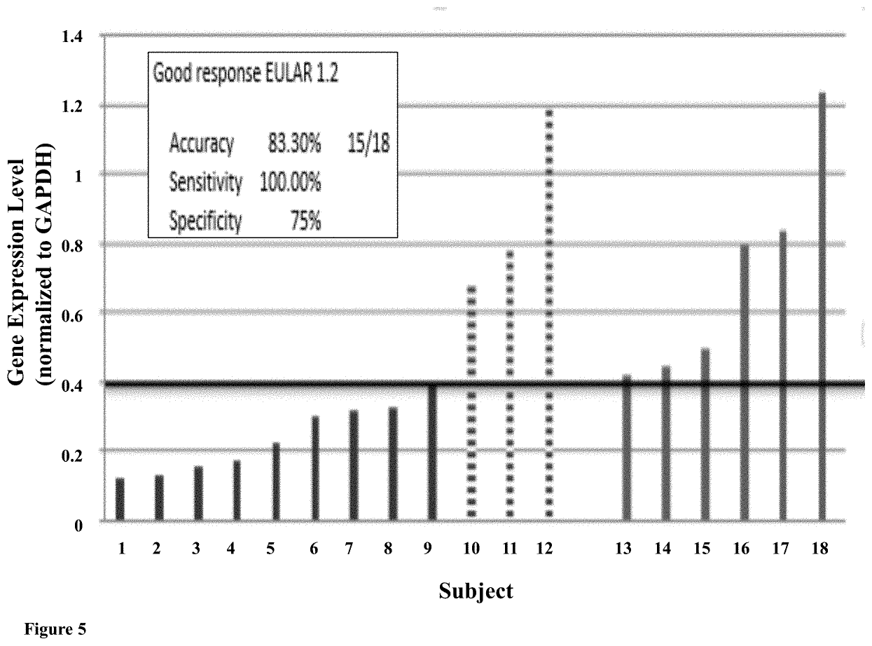

Of the eighteen patients, 6 achieved a good EULAR and 6 achieved a moderate EULAR response. Six patients did not respond to TNFα blocker treatment. Prediction results utilizing the disclosed kit, in comparison to actual clinical evaluation after three months are displayed in FIGS. 4 and 5.

The method and kit of the invention correctly predicted the patients' response to TNFα blockers in 16 of 18 patients (accuracy—89%, specificity—67%, sensitivity—100%) when using the EULAR moderate response criteria as a positive response to blockers and a threshold of 0.25 (for the weighted sum expression level of 12 genes) (FIG. 4) and in 15 of 18 patients (accuracy—83.3%, specificity—75%, sensitivity—100%) when using the EULAR good response criteria as a positive response to blockers and a threshold of 0.4 (for the weighted sum expression level of 12 genes) (FIG. 5) leading to a total prediction accuracy of 83.3-89%. Prediction accuracy, sensitivity and specificity parameters are presented in Tables 2 and 3.

TABLE 2
Prediction accuracy according to
EULAR moderate response criteria

Clinical results according

| | |
|---|---|
| to EULAR moderate | Prediction |

| response criteria | | Responders | Non-responders |
|---|---|---|---|
| Responders | 12 | 12 | 0 |
| Non-responders | 6 | 2 | 4 |

| | |
|---|---|
| Accuracy | 89% |
| Specificity | 67% |
| Sensitivity | 100% |

TABLE 3
Prediction accuracy according to EULAR good response criteria

| | |
|---|---|
| Clinical results according | |
| to EULAR good | Prediction |

| response criteria | | Responders | Non-responders |
|---|---|---|---|
| Responders | 6 | 6 | 0 |
| Non-responders | 12 | 3 | 9 |

| | |
|---|---|
| Accuracy | 83.3% |
| Specificity | 75% |
| Sensitivity | 100% |

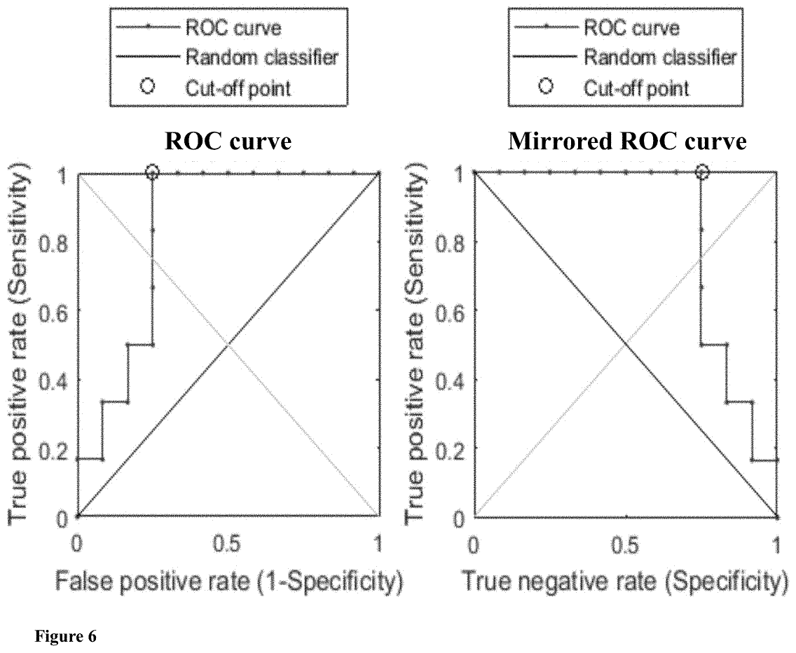

ROC analysis applied to the 2 published datasets yielded an AUC of 0.89. Similarly, ROC analysis applied to the prospective data yielded an AUC of 0.83 with a sensitivity of 100% and a specificity of 75% (FIG. 6).

We calculated the statistical power based on the averages and standard deviation of the weighted sum gene expression of the two groups (responders and non-responders) assuming a two-sample pooled t-test for normally distributed data with unknown standard deviation and equal variances and a ratio of two (12 non-responders and 6 responders) between the groups. The statistical power obtained in the prospective data study was 0.9.

High expression levels of the prediction genes predicted a moderate to good response while low levels were associated with a negative response, corroborating our findings when applying the currently disclosed personal gene expression signature to published data sets.

### Example 3

### Reduced Gene Set

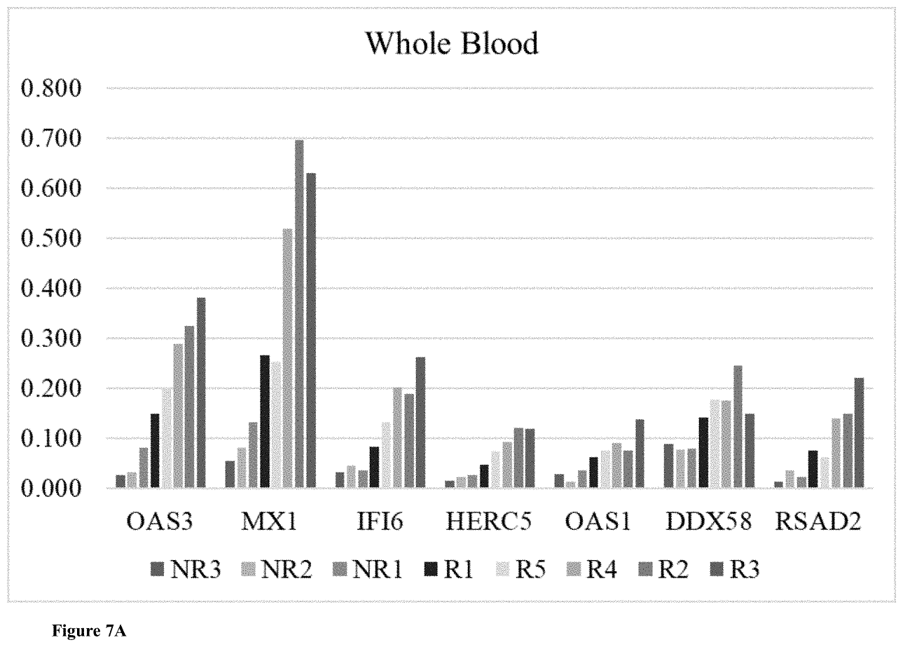

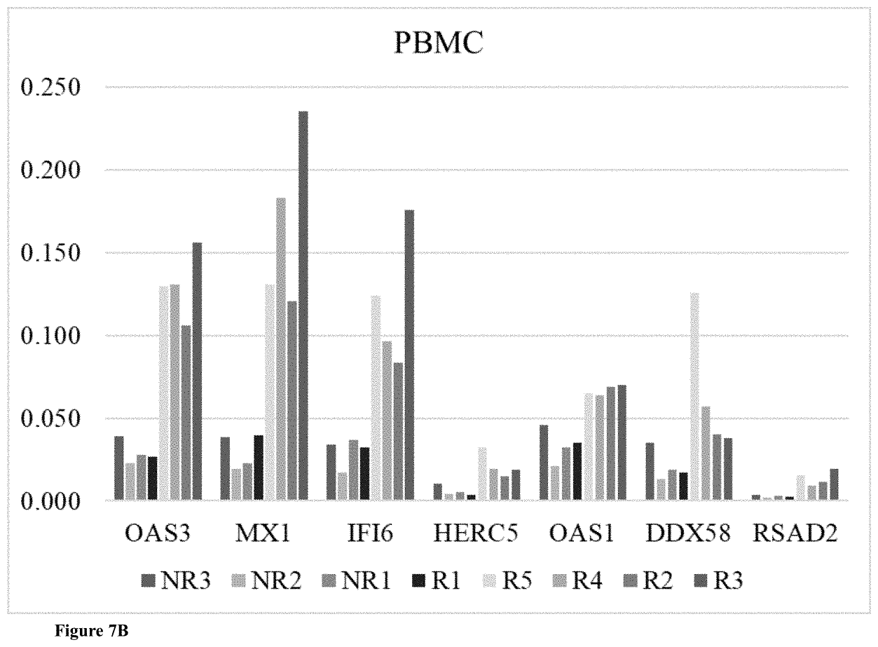

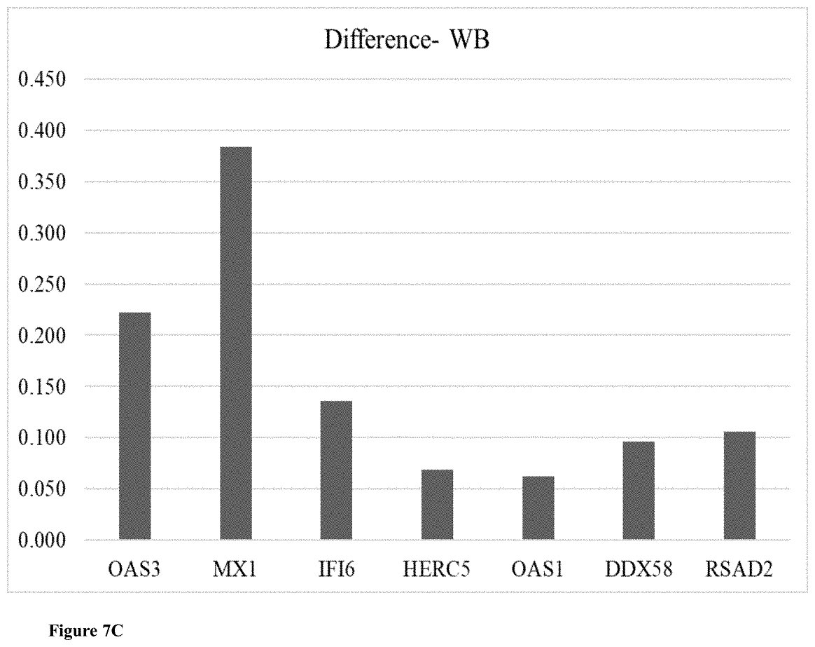

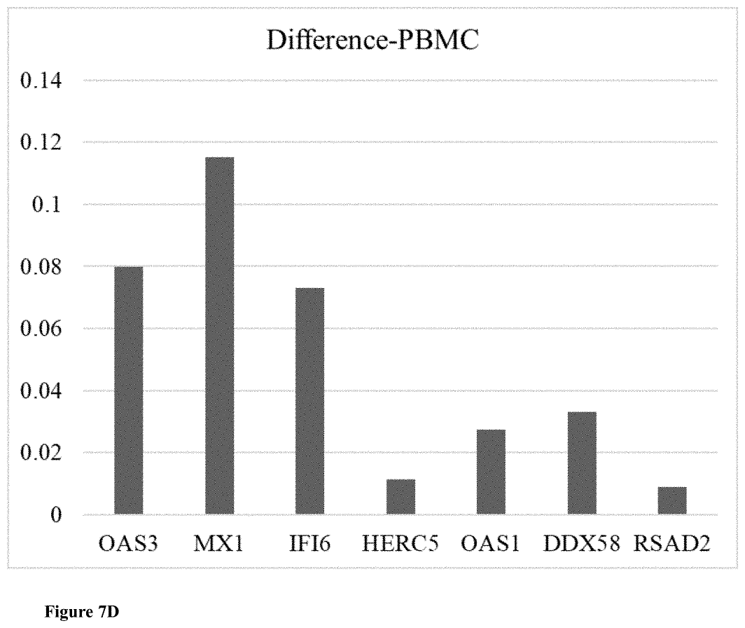

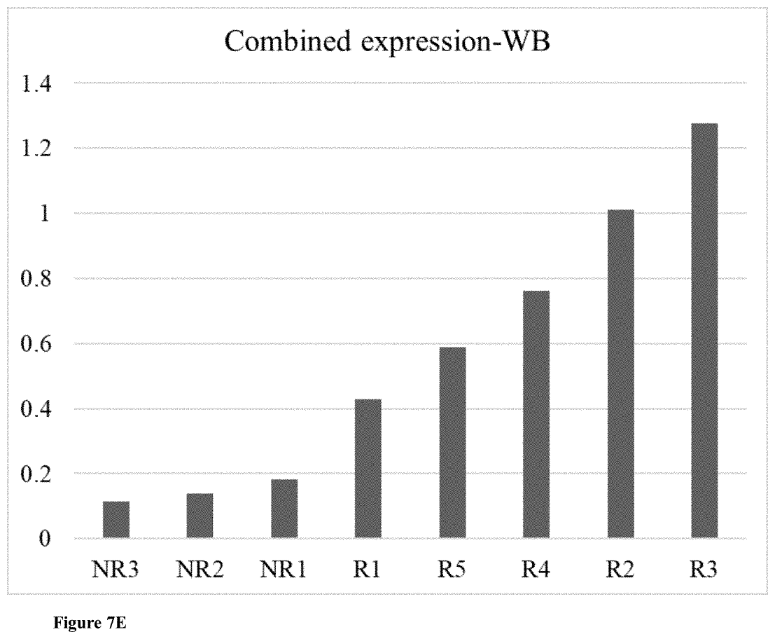

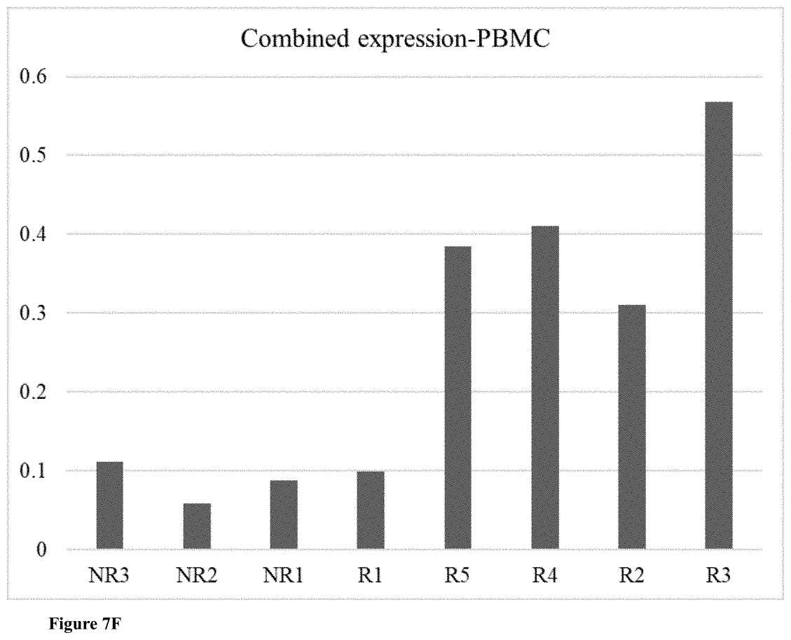

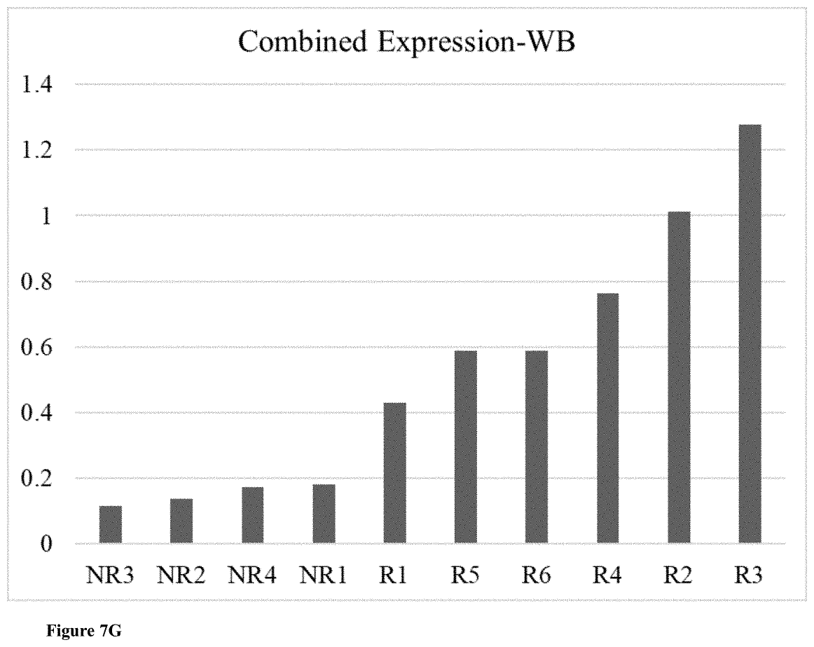

In order to further refine the kits of the invention, blood samples from 4 patients from Ichilov Medical Center in Tel Aviv and 4 blood samples from A.M.C. Medical Center Laboratory LTD. obtained. The clinical response to TNF blockers was known for each sample (5 responders, 3 non-responders). RNA was extracted both from whole blood (WB) by Tempus RNA isolation kits, and from PBMCs isolated from the blood. Each of the 12 genes was examined individually and a very high correlation was found in gene expression for each individual. FIG. 7A shows expression of seven representative genes (OAS3, DDX58, RSAD2, MX1, IFI6, HERC5, OAS1) in the whole blood of the 8 patients. As is readily evident, all genes showed a similar pattern of expression, although some are more highly expressed then others. Further, responders can be easily separated from non-responders although the threshold varies for each gene. Similar results were found when expression in PBMC was examined (FIG. 7B), although whole blood gave more robust results.

Although all seven genes were predictive, by averaging the expression in all responders and subtracting the average expression in all non-responders, the average difference in expression could be calculated. As is seen in FIGS. 7C and 7D (whole blood and PBMCs respectively), three genes, OAS3, MX1 and IFI6 showed by far the largest difference in responders and non-responders. This was true in whole blood and PBMCs. When these 3 genes alone were used in place of all 12 (sum of the expression of the 3 genes) the three non-responders and 5 responders were readily identifiable from the whole blood samples (FIG. 7E). One responder was misidentified when PBMCs were used (FIG. 7F). Addition of two new patient samples to the analysis (1 responder, 1 non-responder), still produced perfect results using the sum of the three genes (FIG. 7G).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for determining and treating a subject suitable to receive anti-TNFα therapy, the method comprising the step of:
a. determining an expression level sum of three genes in a biological sample obtained from the subject, wherein said three genes are
i. MX1
ii. at least one gene selected from IFI6 and OAS3, and
iii. at least one gene selected from the group consisting of: IFI6, OAS1, OAS3, DDX58, RSAD2, IFIT1, IFIT3, IFI44, IFI44L, IFITM3, and HERC5; and
b. generating a diagnosis regarding suitability to receive anti-TNFα therapy wherein:
an expression level sum above a predetermined threshold, is indicative of the subject being suitable to receive anti-TNFα therapy, and
an expression level sum below a predetermined threshold, is indicative of the subject being unsuitable to receive anti-TNFα therapy,
c. treating said subject being suitable to receive anti-TNFα therapy, with anti-TNFα therapy;
thereby treating a subject suitable to receive anti-TNFα therapy.

2. The method of claim 1, wherein said biological sample is peripheral blood mononuclear cell (PBMC), or whole blood.

3. The method of claim 1, wherein said subject suffers from rheumatoid arthritis.

4. The method of claim 1, wherein said anti-TNFα therapy is TNFα blocker therapy.

5. The method of claim 1, wherein said third gene is selected from IFI6, OAS1, OAS3, DDX58, RSAD2, and HERC5.

6. The method of claim 1, wherein said three genes are MX1, IFI6 and OAS3.

7. The method of claim 1, wherein said predetermined threshold is between 0.2-0.5.

8. The method of claim 1, further comprising treating said suitable subjects with anti-TNFα therapy.

9. The method of claim 1, further comprising transmitting the diagnosis to the subject.

10. A kit comprising reagents adapted to specifically determine the expression level of MX1, and at least one of IFI6, and OAS3.

11. The kit of claim 10, comprising reagents adapted to specifically determine the expression level of MX1, IFI6 and OAS3.

12. The kit of claim 10, further comprising at least one reagent adapted to specifically determine the expression level of a gene selected from IFI6, OAS1, OAS3, DDX58, RSAD2, IFIT1, IFIT3, IFI44, IFI44L, IFITM3, and HERC5.

13. The kit of claim 10, further comprising at least one reagent adapted to specifically determine the expression level of a gene selected from IFI6, OAS1, OAS3, DDX58, RSAD2, and HERC5.

14. The kit of claim 10, wherein said reagents are selected from nucleic acid hybridization or amplification reagents, and a plurality of nucleic acid-specific probes or amplification primers.

15. The kit of claim 10, further comprising any one of: (i) detectable tags or labels, (ii) solutions for rendering a nucleic acid susceptible to hybridization, (iii) solutions for lysing cells, (iv) solutions for the purification of nucleic acids, (v) any combination of (i), (ii), (iii), (iv) and (v).

16. The kit of claim 10, further comprising at least one reagent adapted to specifically determine the expression level of at least one house-keeping gene.

17. The kit of claim 10, consisting of:
a. at least one reagent adapted to specifically determine the expression level of MX1
b. at least one reagent adapted to specifically determine the expression level of at least one gene selected from IFI6 and OAS3;
c. at least one reagent adapted to specifically determine the expression level of at least one gene selected from IFI6, OAS1, OAS3, DDX58, RSAD2, IFIT1, IFIT3, IFI44, IFI44L, IFITM3, and HERC5, wherein said gene is not the gene selected in (b); and
d. at least one reagent adapted to specifically determine the expression level of at least one house-keeping gene.

18. The kit of claim 14 or 5, wherein said house-keeping gene is a gene that is not differentially expressed between subjects that respond and subjects that do not respond to anti-TNFα therapy.

19. The kit of claim 10, consisting of:
a. at least one reagent adapted to specifically determine the expression level of MX1
b. at least one reagent adapted to specifically determine the expression level of at least one gene selected from IFI6 and OAS3; and
c. at least one reagent adapted to specifically determine the expression level of at least one gene selected from IFI6, OAS1, OAS3, DDX58, RSAD2, IFIT1, IFIT3, IFI44, IFI44L, IFITM3, and HERC5, wherein said gene is not the gene selected in (b).

20. The kit of claim 19, consisting of at least one reagent adapted to specifically determine the expression level of MX1, at least one reagent adapted to specifically determine the expression level of IFI6 and at least one reagent adapted to specifically determine the expression level of OAS3.
